(19) United States
(12) Patent Application Publication
(10) Pub. No.: US 2016/0066805 A1
(43) Pub. Date: Mar. 10, 2016

(54) ASSEMBLY FOR PROVIDING A LONG-TERM ECG

(71) Applicant: GETEMED MEDIZIN - UND INFORMATIONSTECHNIK AG, Teltow (DE)

(72) Inventors: Michael SCHERF, Berlin (DE); Robert DOWNES, Kleinmachnow (DE); Christian KLERX, Berlin (DE)

(21) Appl. No.: 14/646,710

(22) PCT Filed: May 21, 2014
(86) PCT No.: PCT/EP2014/060442

§ 371 Date: May 21, 2015

(30) Foreign Application Priority Data
May 23, 2013 (DE) 10 2013 209 593.7

(51) Int. Cl. A61B 5/0404; A61B 5/044; A61B 5/0408; A61B 5/0432; A61B 5/00

(52) U.S. Cl. CPC A61B 5/0404; A61B 5/0432; A61B 5/0006; A61B 5/6898; A61B 5/0408; A61B 5/044; A61B 5/684; A61B 5/0024; A61B 5/0022; A61B 2562/222; A61B 2562/08; A61B 2560/0214

(57) ABSTRACT

The invention relates to an assembly for providing a long-term ECG. The assembly comprises a portable ECG device (1), which is designed and intended to record, for a defined or definable time period, an ECG of a patient who can be associated with the ECG device (1), and an application software (2) for the ECG device (1) that can be executed on a mobile device (3) or on a computer. By means of the application software (2), parameters of the ECG device (1) can be set and data recorded by the ECG device (1) can be read out and displayed. Furthermore, the application software (2) is designed to visually instruct an operating person in the electrode placement for an ECG and to detect and evaluate parameters of the ECG device (1) and/or of an ECG cable (41-43) connected to the ECG device (1).

### CROSS-REFERENCE TO A RELATED APPLICATION

This application is a National Phase Patent Application of International Patent Application Number PCT/EP2014/060442, filed on May 21, 2014, which claims priority of German Patent Application Number 10 2013 209 593.7, filed on May 23, 2013.

### BACKGROUND

The present invention relates to an assembly for providing a long-term electrocardiogram (long-term ECG).

It is known to record an electrocardiogram (ECG) over a longer period of time, for example, for 12 hours, 24 hours, or even seven days. Such a long-term ECG is employed, for example, to be able to improve the detection and evaluation of cardiac arrhythmia. It is known to use portable recording devices for recording a long-term ECG. For example, GETEMED Medizin—und Informationstechnik AG in 14513 Teltow, Germany, provides a recorder for recording long-term ECGs under the name of CardioMem CM 4000. This recorder includes a large color display which displays relevant information, including signal curves.

### SUMMARY

An object of the present invention is to provide an assembly for providing a long-term ECG which is characterized by a simple and economical design.

This object is achieved by an assembly for providing a long-term ECG as described herein and an application software program for an ECG device as described herein.

The approach according to the present invention is accordingly characterized in that the assembly according to the present invention comprises two components; on the one hand, a portable ECG device which is designed and provided to record an ECG of a patient, who can be associated with the ECG device, for a defined or definable period of time, and on the other hand, an application software program for the ECG device which is executable on a mobile device or on a computer. It is provided that parameters of the ECG device may be set, and data recorded by the ECG device may be read out and displayed, via the application software program. Furthermore, it is provided that the application software program is designed to visually instruct an operator in the electrode placement for an ECG and in addition, to detect and evaluate parameters of the ECG device and/or of an ECG cable connected to the ECG device.

The approach according to the present invention is thus based on the idea of no longer carrying out the operation of an ECG device on the device itself, but rather via an application software program which is executable on a mobile device or on a computer. Such an application software program is often referred to as an “app.” Since the operation and depiction of the data recorded by the ECG device is carried out via the application software program, the ECG device itself may be designed in a simple manner, in particular without its own display. The operation, the depiction of the signal curves of the recorded ECG data, and the attachment of an ECG cable to a patient are all controlled via the application software program.

Thus, according to one exemplary embodiment of the present invention, it is provided that the application software program is designed to set the duration of an ECG recording as a parameter of the ECG device. For example, the duration of an ECG recording may be set to a period of time of 12 hours, 24 hours, or seven days. Another exemplary embodiment of the present invention provides that the application software program is designed to set the type of the battery to be used for a recording, if the ECG device includes different battery types, for example, a rechargeable battery and a replaceable battery.

In addition, according to one exemplary embodiment, the application software program is designed to detect parameters of the ECG device or of an ECG cable connected to the ECG device. Such parameters are, for example, the serial number of an ECG cable which is connected to the ECG device and which is used for an ECG recording, the type of an ECG cable connected to the ECG device (three-wire, five-wire, or seven-wire), the contact status of the electrodes of an ECG cable, the number of channels which are detected via an ECG recording, and the battery status of at least one battery. The detection of such parameters completes the information obtained while recording an ECG and makes it possible to detect sources of error if necessary. For example, knowledge of the serial number of an ECG cable used during an ECG recording makes it possible to locate the ECG cable in the event of erroneous data and to check whether it is possibly defective. The detection of the contact status of the electrodes of the ECG cable also makes it possible to identify causes of erroneous data and to provide instruction functions for correcting a possibly poor contact of the electrodes with the patient.

In another exemplary embodiment, the application software program is also designed to transmit information to the ECG device which is not directly used for operating the ECG device, but which provides it with information which is important for an ECG recording. Such information, for example, includes patient data, i.e., data, for example, about the identity, gender, and age of a patient, and data about an operator who attaches an ECG cable to a patient. This data is of course also stored in the application software program or on the mobile device or computer on which the application software program is executed. Its additional provision in the ECG device allows linking the recorded ECG data already in the ECG device to such information.

One advantageous embodiment of the assembly according to the present invention provides that different ECG cables may be connected to the ECG device, and that the ECG device automatically detects the type of the connected ECG cable and provides this information to the application software program. For example, the ECG device detects whether the connected ECG cable is a three-wire ECG cable, a five-wire ECG cable, or a seven-wire ECG cable. The automatic detection of a connected ECG cable and the transfer of this information to the application software program make it possible, for example, to provide instructions in the application software program for placing electrodes on a patient with respect to the specific ECG cable which is connected.

Thus, in another exemplary embodiment, the present invention provides that the application software program is designed to instruct an operator during the electrode placement for an ECG and in addition, to detect and evaluate parameters of the ECG device and/or of an ECG cable connected to the ECG device. It may be provided that with the aid of the application software program, the places on the human body at which electrodes of the detected ECG cable are to be attached to the human body are schematically indicated on a display (for example, the display of a mobile device or a computer screen). The depiction is preferably carried out using color coding corresponding to a color coding of the detected ECG cable. ECG cables generally have a color coding for facilitating the attachment and placement of the electrodes.

According to another aspect of the present invention, it is provided that the application software program is designed to carry out a checking function with respect to a correct contacting of the electrodes of an ECG cable with the body of a patient. For this purpose, it may, for example, be provided that it is ascertained via an evaluation of electrical parameters, for example, the voltage drop across each electrode of an ECG cable, whether an electrode is correctly attached to the human body. If this is not the case, the application software program may generate a warning message to an operator that correct contacting of the electrodes with the patient has not yet been established.

An additional aspect of the assembly according to the present invention provides that the application software program comprises a device management which allows the detection and possibly the management of multiple ECG devices. Such a device management makes it possible in particular to find and connect ECG devices, and to manage devices which have already been detected and to select them with respect to a measurement to be carried out.

An additional aspect of the assembly according to the present invention provides that the application software program comprises a patient management which allows the detection of multiple patients. Such a patient management comprises, for example, a table of the recorded patients including suitable patient information such as the name, gender, age, and identification number, and the option of adding additional patients or deleting patients who have already been recorded.

According to an additional aspect of the assembly according to the present invention, the application software program also comprises electrode placement instructions for an operator which provide for carrying out certain procedure steps in a defined sequence. For example, it is provided that the electrode placement instructions allow an operator to select a patient from a patient list and to associate the patient with an ECG device which is included in an ECG device list of the application software program. In addition, an electrode placement is instructed and an ECG recording is subsequently started. The instructions for the electrode placement may be carried out visually on the display screen of the mobile device or the computer on which the application software program is installed, for example, via a schematic representation of the upper body of the patient and the points at which the electrodes of an ECG cable are to be placed.

Furthermore, it may be provided that the electrode placement instructions are designed in such a way that a patient on the patient list is deleted from the list as soon as an ECG recording is started for the patient. This enables an operator to ensure in a simple manner that all patients on a patient list are processed sequentially with respect to the attachment of an ECG cable and the start of an ECG recording.

In one exemplary embodiment, the application software program is designed in such a way that it provides a graphical user interface which comprises multiple windows which provide functions for the device management, the patient management, and the electrode placement.

As already mentioned, the approach according to the present invention is based on the idea that the operation of the ECG device is carried out via the application software program. Accordingly, it is advantageously provided that the ECG device itself has no display of its own, and recorded ECG data may be depicted only with the aid of the application software program.

It may thus be provided that the ECG device has only a single control element, namely, an control element for switching the ECG device on and off. At least one additional function may be associated with the control element, for example, the setting of a marking in an ECG by a patient at a desired point in time during the recording of an ECG. Thus, if the patient subjectively senses an abnormality, he/she may operate the control element and thereby set a marking in the ECG, so that a physician may check at a later time whether the subjective sensation experienced by the patient correlates with actual abnormalities in the ECG at the observed point in time.

According to one advantageous embodiment of the present invention, the ECG device thus has only a single control element which is used for switching the ECG device on and off and which additionally, for example, enables the setting of a marking in the ECG by a patient. The other operator control functions are implemented via the application software program.

The application software program may be an application software program for mobile devices, in particular for smartphones and tablet computers.

According to another exemplary embodiment of the assembly according to the present invention, it is provided that the application software program communicates with the ECG device wirelessly, for example, via Bluetooth technology. For this purpose, corresponding Bluetooth modules are configured in the ECG device and in the mobile device or the computer on which the application software program is installed.

It lies within the scope of the present invention that the application software program is designed to transmit data received by the ECG device and/or data derived from such data (i.e., data which have already been evaluated or pre-evaluated) to a telemedicine center and/or to an evaluation center for storing the data and further evaluating it as necessary. This may be carried out automatically.

An additional embodiment of the present invention provides that the ECG device is a long-term ECG recorder and that the application software program is designed to retrieve all ECG information in streaming mode during the ECG recording and/or to be able to carry out at least one setting on the recorder. This makes it possible to depict the ECG in streaming mode during the ECG recording, for example, for diagnostic purposes or for checking the signal quality.

In particular, it may be provided that all parameters of the recorder and the ongoing ECG recording may be displayed with the aid of the application software program and/or that all parameters of the recorder may be changed and/or corrected during the ongoing ECG recording using the application software program. According to one embodiment variant, the application software program may be designed to be able to interrupt the ECG recording at any time and to subsequently carry out a function and/or quality check of the recorder.

An additional embodiment provides that error messages generated by the recorder (for example, a detached electrode or an empty battery) are displayed and indicated by the application software program.

The present invention thus considers not only the so-called hookup, i.e., the procedure for preparing an ECG recording, but also provides additional options during an ECG recording. This is associated with several advantages:

a) The signal quality may be checked during the recording, making it possible to detect malfunctions. It is also possible to detect detached electrodes.

b) The ECG may be observed at all times. This is of great value particularly in the clinical setting in the case of acute symptoms. The disadvantage of a long-term ECG over one, two, or seven days is that acute cardiac arrhythmia which must be treated immediately is not detected.

c) Checking the condition of the patient is possible even after interruption of the recording, for example, for performing an MRT.

d) The parameters of the recorder may be corrected during the recording.

e) A complete function check of the recording system is may be carried out.

The present invention also relates to an application software program for an ECG device which is executable on a mobile device or on a computer, wherein the application software program is designed to be employed in an assembly for providing a long-term ECG according to claim 1 or to interact with a portable ECG device corresponding to the assembly of claim 1.

### BRIEF DESCRIPTION OF THE DRAWINGS

The present invention is described in greater detail below based on multiple exemplary embodiments, with reference to the figures.

FIG. 1 shows an exemplary embodiment of an assembly for providing a long-term ECG which comprises a portable ECG device and an application software program, wherein the components of the ECG device are depicted in the block diagram.

FIG. 2 shows a perspective view of an exemplary embodiment of a portable ECG device.

FIG. 3 shows a display screen view provided by an application software program on a mobile device or computer depicting a first step for an electrode placement.

FIG. 4 shows a display screen view provided by an application software program on a mobile device or computer depicting a second step for an electrode placement.

FIG. 5 shows a display screen view provided by an application software program on a mobile device or computer depicting ECG signals, devices, and patient information, and a schematic representation of the electrodes of an ECG cable to be placed.

FIG. 6 shows a representation corresponding to FIG. 5, wherein ECG signals are present for three channels and a query is made whether a recording is to be started.

FIG. 7 shows a display screen view provided by an application software program on a mobile device or computer for an operator for instructing an electrode placement.

FIG. 8 shows a display screen view provided by an application software program on a mobile device or computer depicting a first operator interface for the device management.

FIG. 9 shows a display screen view provided by an application software program on a mobile device or computer depicting a second operator interface for the device management.

FIG. 10 shows a display screen view provided by an application software program on a mobile device or computer depicting a third operator interface for the device management.

FIG. 11 shows a display screen view provided by an application software program on a mobile device or computer depicting an operator interface for the patient management.

### DETAILED DESCRIPTION

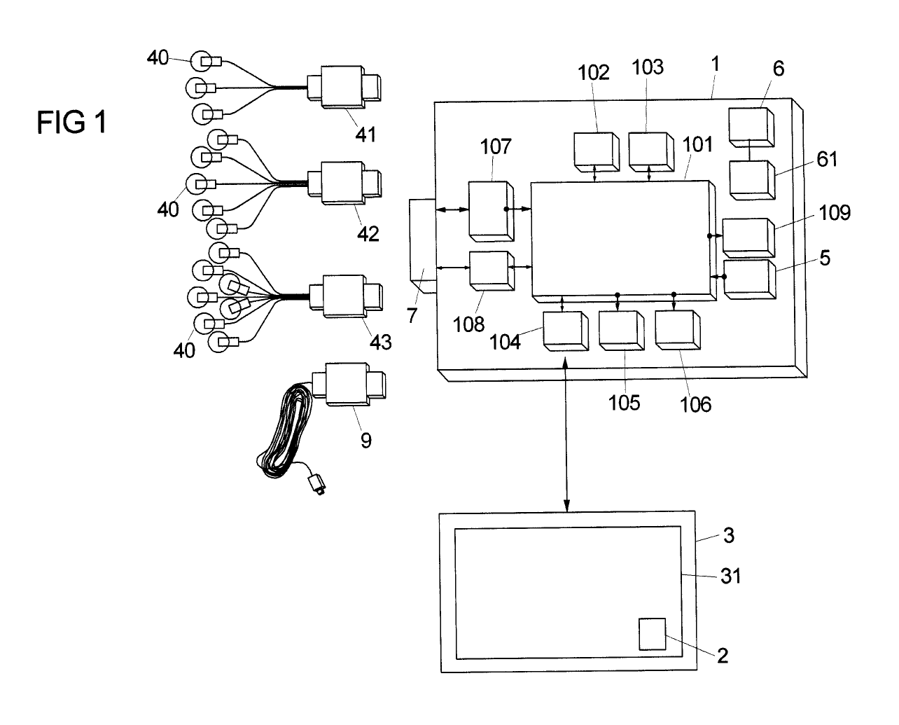

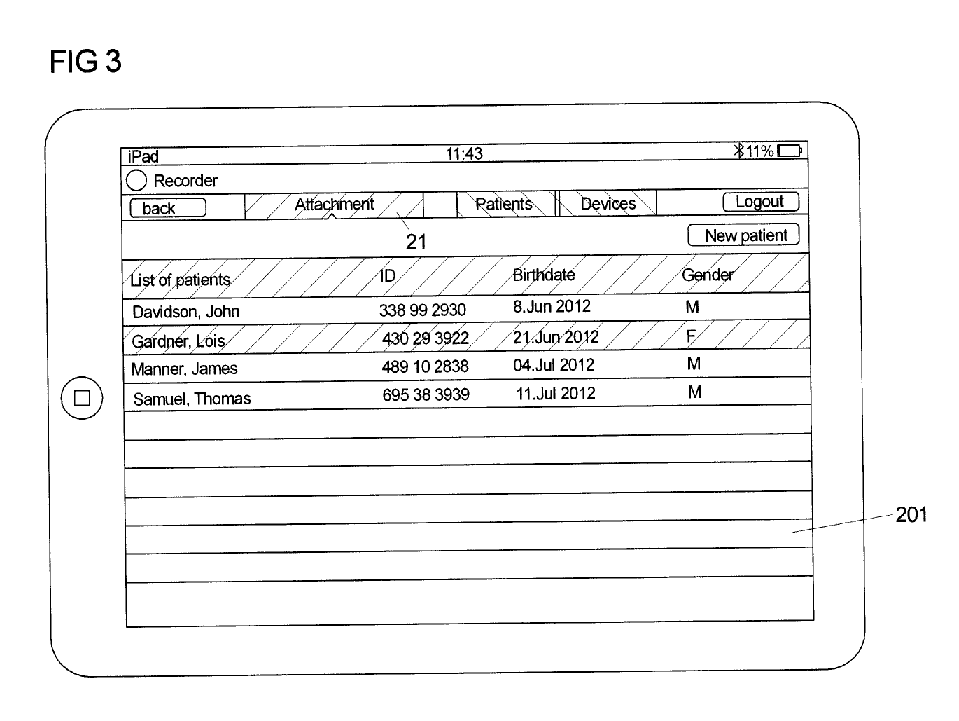

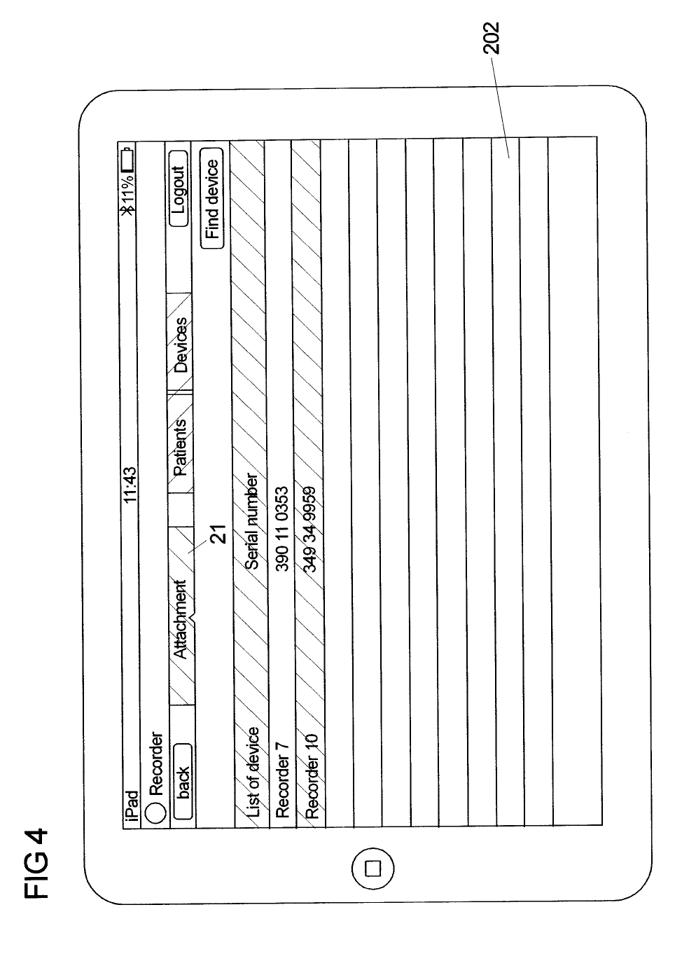

FIG. 1 shows an exemplary embodiment of an assembly for providing a long-term ECG. The assembly comprises a portable ECG device 1, to which various ECG cables 41-43 may be connected, and an application software program 2. The application software program is executable on a device 3 which is a mobile device or a computer and which has a display screen 31. The representation of FIG. 1 depicts the application software program 2 as an icon on the display screen 31. When the application software program 2 is opened, the application software program 2 provides various user interfaces, which will be explained based on FIGS. 3 through 11.

In the depicted exemplary embodiment, the mobile device 3 is a tablet PC. However, the application software program 2 may also be installed and executed on any other mobile device, for example, a smartphone, or alternatively on a computer, in particular a desktop computer.

The ECG device 1 comprises a microprocessor 101, a data memory 102, a real-time clock 103, a Bluetooth interface 104, two LEDs 105, 106, an amplifier 107 for ECG signals, a protocol converter 108 for USB, a piezoelectric element 109, an on/off control element 5, a receiving jack 7, a battery 6, and an energy management unit 61 which is associated with the battery 6, as hardware components.

The Bluetooth interface 104 is used for mobile communication with the mobile device or computer 3 or the application software program 2 installed on it. For this purpose, the device 3 also includes a Bluetooth interface (not separately depicted) in the conventional manner.

The various ECG cables 41, 42, 43 may be connected to the receiving jack 7. In addition, a USB cable 9 may alternatively also be connected, for example, in order to read out the information stored in the memory 102 in a manner other than via the Bluetooth interface 104.

The various connectable ECG cables 41 to 43 include a three-wire ECG cable 41, a five-wire ECG cable 42, and a seven-wire ECG cable 43. The cables 41 to 43 each comprise electrodes 40 on their ends corresponding to the number of wires in each cable. The electrodes 40 are attached to a human body by an operator. The cables are preferably color-coded, as is known per se.

After pre-amplification in the amplifier 107, the signals provided by an ECG cable 41 to 43 are stored in the data memory 102, and after a recording is completed, they are transmitted via the Bluetooth interface 104 to the device 3 for display and evaluation with the aid of the application software program 2. The operation of the ECG device 1 is carried out via the application software 2, as explained in detail below.

The protocol converter 108 for USB is used if the USB cable 9 is connected.

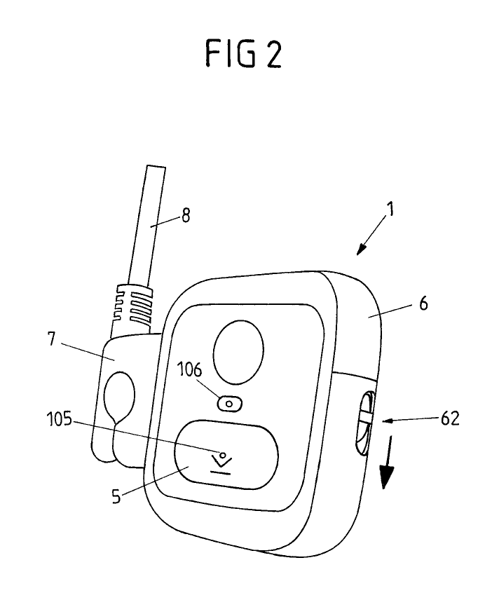

With respect to the function of the LEDs 105, 106, the on/off control element 5, and the battery 6, reference is also made to the perspective view of an ECG device 1 in FIG. 2.

The control element 5 is used for switching the device 1 on and off. For example, it may be provided that if the control element 5 is pressed for more than three seconds, the device is started, and if the control element 5 is pressed again for more than three seconds, the device is switched off. In addition, the control element 5 may be associated with at least one additional function, in particular, the generation of a marking in an ECG which is currently being recorded. For this purpose, it may, for example, be provided that by pressing the control element 5 for less than three seconds, such a marking is set and recorded in the ECG. It may also be provided that by pressing the control element 5 for, for example, more than three seconds during an ECG recording, a wireless data transmission via Bluetooth is activated, and that by pressing the control element 5 after ending an ECG recording, a download of the recorded data is activated. However, such a download may also be initiated via the application software program 2.

It is to be noted that the ECG device 1 includes only a single control element 5. The control and operation of the ECG device 1 is carried out via the application software program 2.

The LED 105 constitutes a status LED. This LED may, for example, assume two different colors, for example, the colors green and yellow. The green color indicates, for example, that the device is ready for recording, and blinking during the recording indicates, for example, that the recording is proceeding correctly. A switch to the other color, for example, yellow, indicates, for example, that problems exist; for example, a previous ECG recording has not yet been deleted, or the battery must be replaced or charged. Other information may also be encoded by the LED 105 and its colors. For example, a slowly blinking yellow light indicates that the battery is weak, and/or blinking alternately between a green color and a yellow color indicates that there is a defect and that service is required.

The LED 106 indicates the status of the electrode placement. It also has two colors, for example, green and yellow. If the electrodes of a connected ECG cable 41 to 43 are correctly attached to a patient, it illuminates green. If an electrode is not attached or is attached incorrectly, it illuminates yellow. In one embodiment variant, the LED 106 has no significance during the recording. Alternatively, this LED may indicate an error message during the recording; for example, blinking alternately between a green color and a yellow color indicates that a there is a defect and that service is required.

In addition, a buzzer may be integrated into the LED device 1 which, for example, emits warning signals in the event of the occurrence of a problem. The buzzer may, for example, be provided by the piezoelectric element 109 of FIG. 1.

An additional feature of the present invention is an automatic pacemaker pulse detection, i.e., the device 1 automatically detects whether pacemaker pulses are present. These pulses are then stored in the ECG data stream as markers (and later evaluated) and depicted as a small dash on the tablet display (above the ECG curve). As a result, when attaching the electrodes, the operator is able to discern whether the pacemaker pulses were correctly detected.

As is apparent from FIG. 2, a battery compartment is provided for accommodating one or multiple batteries 6, which may be closed via a latch 62. It may thus be provided that the battery compartment includes both replaceable batteries and rechargeable batteries. In the case of rechargeable batteries, it may be provided that the ECG device 1 itself includes a connector for recharging a rechargeable battery (not shown).

In the representation of FIG. 2, an ECG cable is plugged into the receiving jack 7, in which the protruding cable bundle 8 leading to the electrodes 40 is apparent.

As shown in the above descriptions, the ECG device 1 includes no control elements which enable a direct operation of the ECG device 1. In particular, the single control element 5 is not suitable for starting an ECG recording and determining the duration of an ECG recording. The ECG device 1 also includes no indication or display which would allow a representation of the recorded ECG data.

Alternatively, it may be provided that the ECG recording is started without the tablet PC 3. Thus, it may be provided that the ECG device has a reliability feature which it starts automatically after approximately eight minutes if the electrodes are attached to the body and the ECG memory is empty. The reason for this is that the operator may prepare the patient but forget to start the recording.

It is provided that parameters of the ECG device 1 may be set via the application software program 2, and data recorded by the ECG device 1 may be read out and displayed with the aid of the application software 2. A data transmission between the ECG device 1 and the application software program 2 or the device 3 on which the application software program 2 is executed is carried out wirelessly, as described in the depicted exemplary embodiment, via Bluetooth technology.

The application software program 2 provides a user interface on the display screen 31 of the device 3 on which it is executed, which provides information to an operator and which enables an interaction by the operator with the application software program. The user interface comprises various windows which an operator may select and which enable an interaction with an operator corresponding to different functionalities.

These different functionalities, with which corresponding windows and sub-windows of the graphical user interface are associated, are electrode placement instructions for preparing and starting an ECG measurement on a patient, a patient management, and a device management. These three different functionalities are indicated in the graphical user interface by the “Attachment,” “Patients,” and “Devices” tabs, using corresponding windows.

FIGS. 3 through 8 show windows of the functionality of an electrode placement for preparing and starting an ECG recording, indicated by the “Attachment” tab.

For example, in the “Attachment” tab 21 in FIG. 3, a window 201 of the application software program 2 is opened which contains a list of the patients on which an ECG recording is to be carried out. In the list, the name of the patient, an identification number (ID), the date of birth, and the gender are specified for each patient. There is also the option of adding an additional patient. An operator may select one of the patients by, for example, scrolling or touching the display screen. In the exemplary embodiment under consideration, the second patient in the depicted list is selected.

After selecting a patient in the window 201, a window 202 is provided in which a list of the available ECG devices is displayed. Another ECG device may be added as necessary. The operator may select one of the displayed devices for further action.

A display screen 203 now opens in the “Attachment” tab or in the “Attachment” module, which depicts the measured ECG signal for one or multiple channels, and which displays information about the patient and the device, and which includes a schematic representation of a human upper body with a color-coded specification of the areas on the human body to which the electrodes of the ECG cable are to be connected. This representation thus provides the operator with a visual, color-coded guide for the electrode placement.

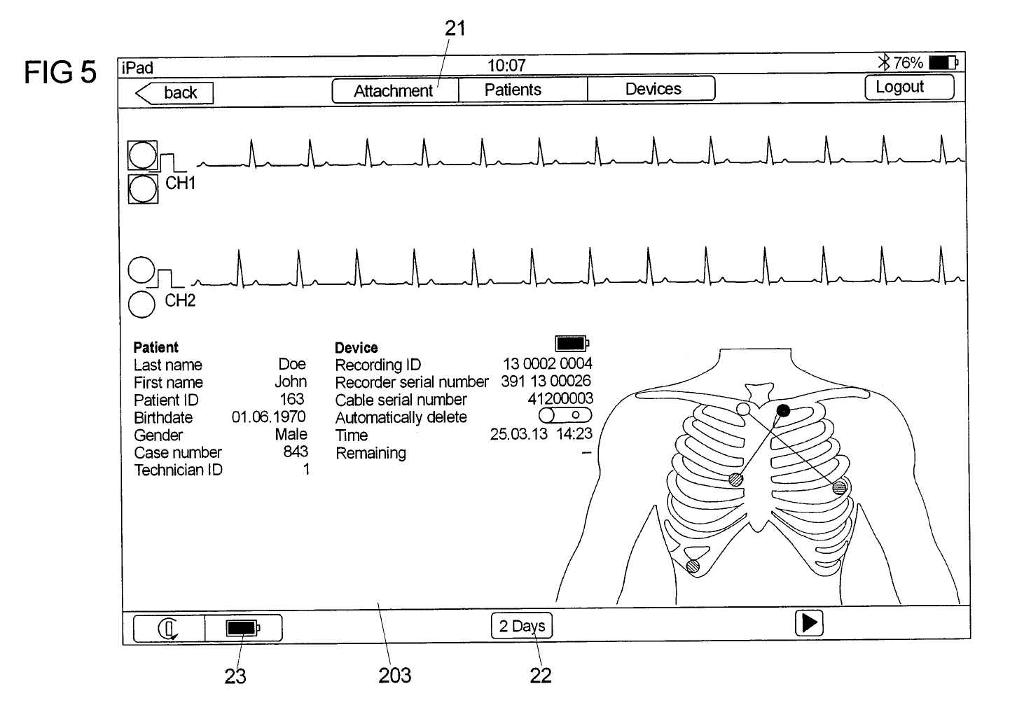

The ECG device 1 automatically detects the type of cable 41 to 43 which is plugged into its receiving jack 7. In particular, the device thus automatically detects whether the connected cable is a three-wire cable, a five-wire cable, or a seven-wire cable. This information is transferred to the application software program 2. The representation on the display screen 303 varies with respect to the electrodes to be placed, depending on which type of cable is used for the current ECG measurement. In the exemplary embodiment of FIG. 5, instructions for a five-wire ECG cable are depicted.

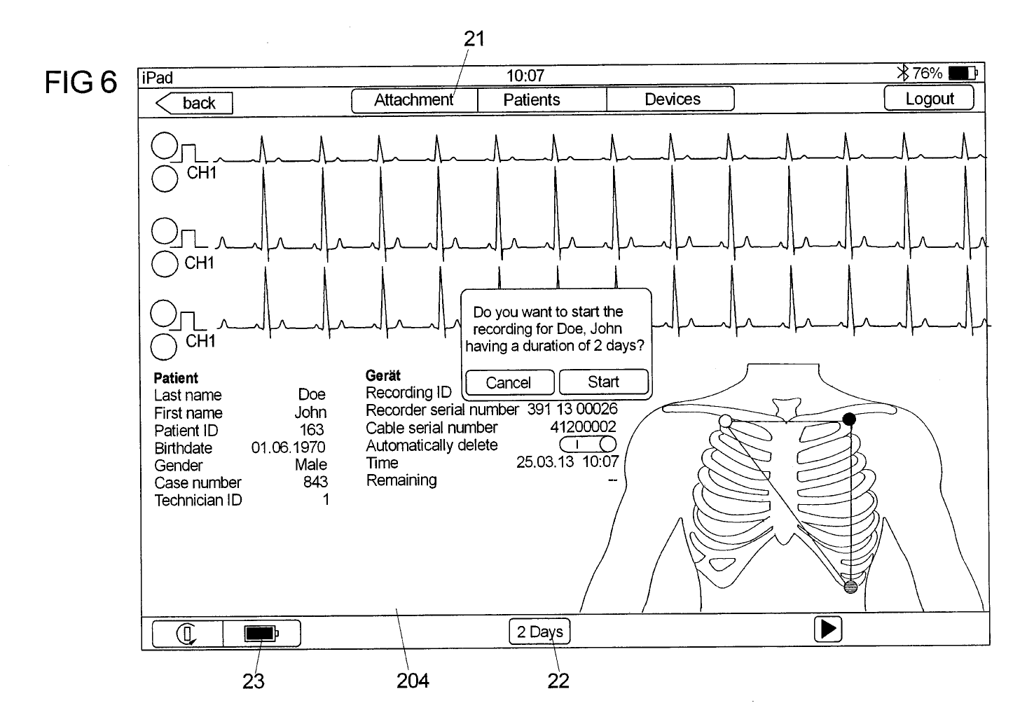

FIG. 6 shows a window 204 which essentially corresponds to the window 203 of FIG. 5, but which differs from the window 203 of FIG. 5 in three respects. Firstly, the connected ECG cable is a three-wire cable; therefore, in the schematic representation of the upper body in the window 204, only three contact points for contacting the electrodes are depicted. Secondly, in the embodiment variant according to FIG. 6, three (interdependent) channels are recorded, instead of two. Thirdly, the attachment of the electrodes to a patient has already progressed far enough that ECG signals are present for each of the three channels. Accordingly, an ECG recording may now be started, as queried by the window depicted in FIG. 6.

In both windows 203, 204 of FIGS. 5 and 6, the duration of an ECG recording may be set by operating a graphical control element 22. The duration is, for example, 12 hours, 24 hours, 48 hours, or seven days.

If multiple battery types are available, a battery type may be selected via the graphical control element 23. Since alkaline disposable batteries have a rated voltage of 1.5 V and rechargeable NiMH batteries have a rated voltage of only 1.2 V, this selection is important for correctly detecting when a battery is exhausted.

Furthermore, it may be provided that the ECG device 1 and/or the application software program 2 or the device 3 checks, based on the battery voltage, whether the selected duration of an ECG recording is actually achievable. If not, a corresponding warning message is displayed on the PC/tablet 3, preferably at the moment at which it is attempted to start a recording.

In addition, in another variant, it is possible to set the number of channels with which the recording is to be carried out via control elements, which are not depicted. The visual instructions for placing electrodes are correspondingly adapted as a function of the number of channels and the detected type of the ECG cable.

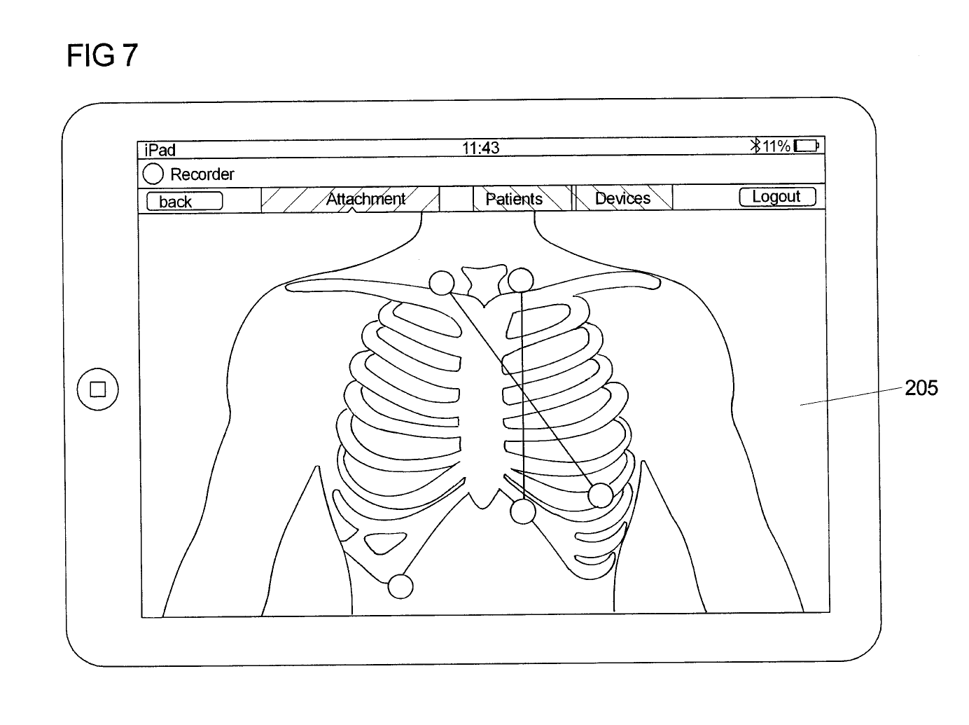

According to FIG. 7, the operator may activate a window 205 which comprises only the visual instructions for the operator for attaching the electrodes of the ECG cable, the corresponding contact points being depicted in color on a schematically represented human upper body.

After an ECG measurement has been started for a selected patient, the relevant person disappears from the list to be processed in the “Attachment” function group according to the window 201 of FIG. 1, so that the operator sees only patients in the list who are still to be processed with respect to a correct electrode placement and the start of an ECG measurement. However, the patients do not disappear from the actual patient management, which may be retrieved via the “Patients” tab.

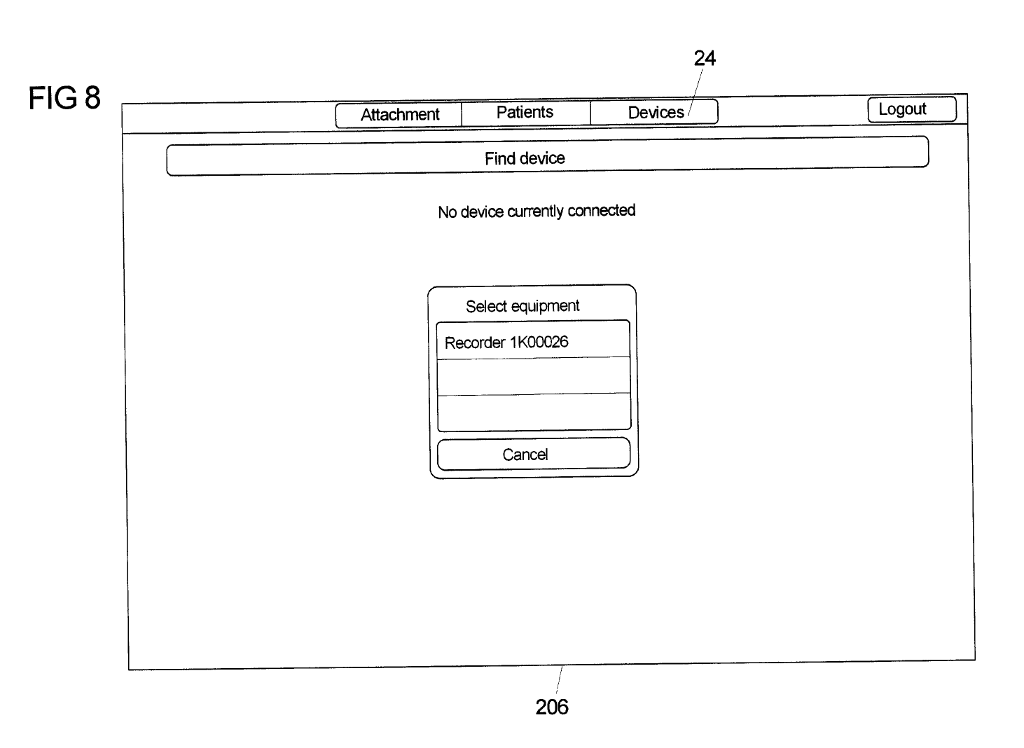

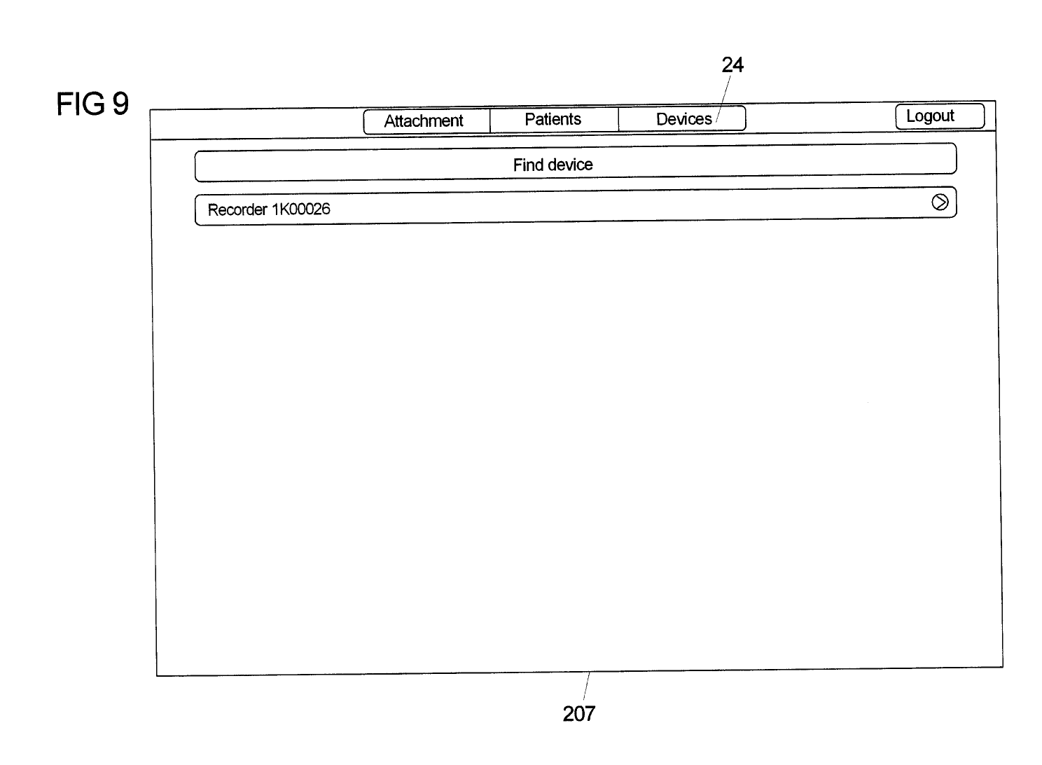

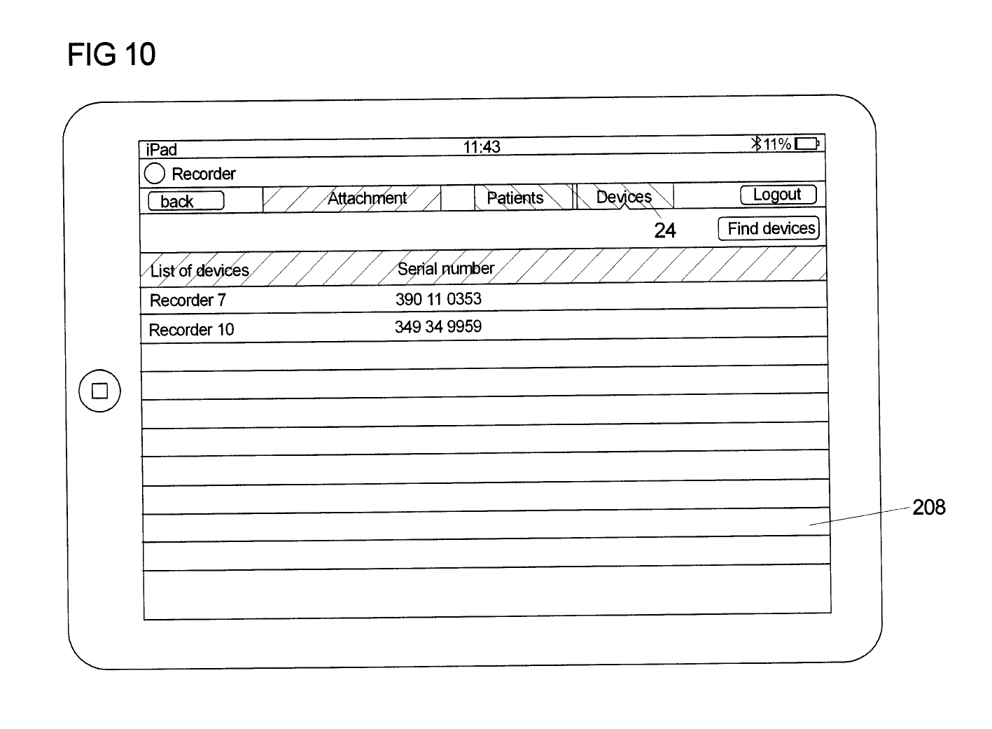

FIG. 8 shows a window 206 which is associated with the functionality of the device management and accordingly with the “Devices” tab 24. According to the window 206, no device is currently registered in the device management; therefore, a device is searched for and made available for connection. Subsequently, according to FIG. 9, a detected device is added to the device management, so that it is available in the event that the electrode placement function group is called. In the same manner, other devices may be added or deleted. FIG. 10 shows a corresponding window 208, in which the device management comprises two devices, for each of which a name and a serial number have been specified.

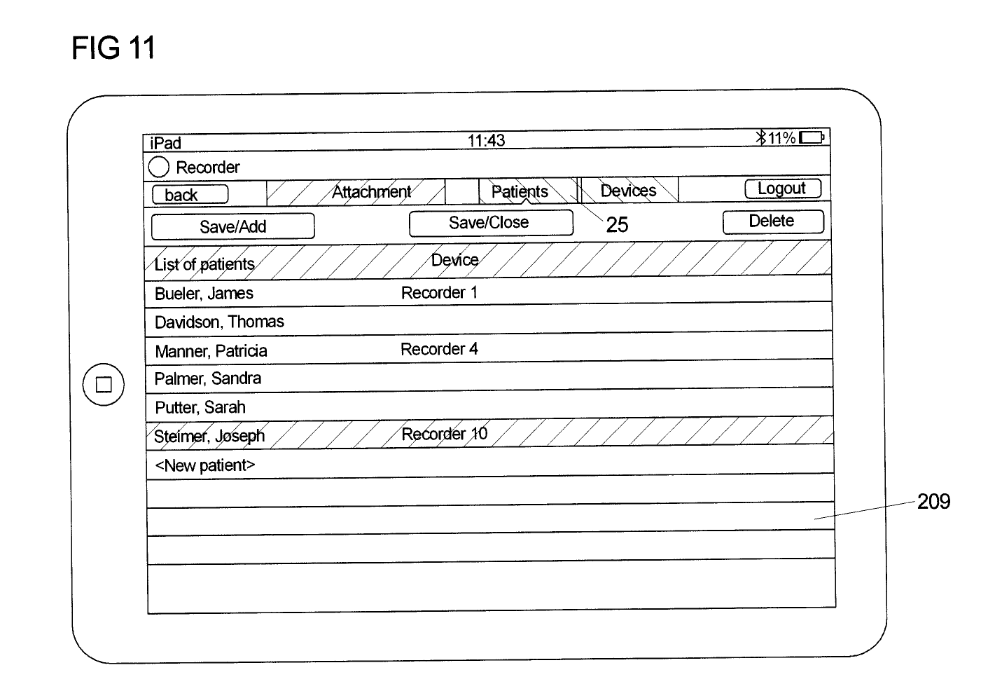

FIG. 11 shows a window 209 of the graphical user interface provided by the application software program 2 of the patient management functionality corresponding to the “Patients” tab 25. The window 209 shows a list of the managed patients including the name and possibly the associated ECG device. Additional patients may be added or deleted. When a patient is selected, an additional window opens (not shown separately), into which personal data such as the name, age, gender, and identification number may be entered.

It is to be noted that additional information windows and method substeps may be provided in particular with the functionality of the electrode placement instructions. Thus, for example, it may be provided that in the event that a check test, which is integrated into the ECG device 1 and/or which is carried out via the application software program 2, demonstrates that not all electrodes 40 of a connected ECG cable 41 to 43 are correctly connected to the human body, this information is passed to the application software program 2 and displayed to the operator as part of a warning message, for example, in a window which opens. From among the electrodes in question, an electrode which is not correctly placed may also be depicted graphically, for example, via blinking of the relevant electrode.

The embodiment of the present invention is not limited only to the presently depicted exemplary embodiments, which are to be understood only to be exemplary. It may thus be provided that the ECG device 1 is configured differently than described in FIGS. 1 and 2.

The design and content of the windows of the graphical user interface may also be designed in a different manner.

1.-31. (canceled)

32. An assembly for providing a long-term ECG which comprises:
a portable ECG device which is designed and provided to record an ECG of a patient, who is able to be associated with the ECG device, for a defined or definable period of time, and
a mobile device or a computer,
wherein the portable ECG device on the one hand and the mobile device or the computer on the other hand are designed to communicate wirelessly with each other via corresponding interfaces,
wherein an executable application software program for the ECG device is installed on the mobile device or on the computer,
wherein parameters of the ECG device may be set, and data recorded by the ECG device may be read out and displayed, via the application software program,
wherein the application software program is designed to visually instruct an operator in the electrode placement for an ECG and, in addition, to detect and evaluate parameters of the ECG device and/or of an ECG cable connected to the ECG device, and
wherein the ECG device is a long-term ECG recorder and the application software program is designed to retrieve ECG information in streaming mode during the ECG recording and to display the ECG in streaming mode during the ECG recording.

33. The assembly as claimed in claim 32, wherein the application software program is designed to detect at least one of the following parameters of the ECG device or of an ECG cable connected to the ECG device:
the type of an ECG cable connected to the ECG device,
the contact status of the electrodes of an ECG cable,
the number of channels which are detected during an ECG recording,
a battery status.

34. The assembly as claimed in claim 32, wherein the application software program is designed to detect the serial number of an ECG cable connected to the ECG device which is used for an ECG recording.

35. The assembly as claimed in claim 32, wherein different ECG cables may be connected to the ECG device, in particular, a three-wire ECG cable, a five-wire ECG cable, and a seven-wire ECG cable, and wherein the ECG device automatically detects the type of the connected ECG cable and provides this information to the application software program.

36. The assembly as claimed in claim 32, wherein the application software program is designed to schematically depict the places on the human body at which electrodes of the detected ECG cable are to be attached to the human body on a display, and wherein the depiction on the display (31) is color-coded corresponding to a color coding of the detected ECG cable (41-43).

37. The assembly as claimed in claim 32, wherein the application software program comprises a patient management which allows the detection of one or multiple patients.

38. The assembly as claimed in claim 32, wherein the application software program comprises electrode placement instructions for an operator which provide for carrying out certain method steps in a defined sequence with respect to the attachment of electrodes of an ECG cable to a patient.

39. The assembly as claimed in claim 38, wherein the electrode placement instructions are designed in such a way that a patient may be selected from a patient list and may be associated with an ECG device from an ECG device list, and subsequently, an electrode placement may be instructed and an ECG recording may be started.

40. The assembly as claimed in claim 32, wherein the application software program is an application software program for mobile devices, in particular for smartphones and tablet computers.

41. The assembly as claimed in claim 32, wherein the application software program communicates with the ECG device wirelessly, for example, via Bluetooth technology.

42. The assembly as claimed in claim 32, wherein the application software program is designed to transmit data received by the ECG device and/or data derived from such data to a telemedicine center and/or an evaluation center.

43. The assembly as claimed in claim 32, wherein the application software program is designed to be able to carry out at least one setting on the ECG device during the ECG recording.

44. The assembly as claimed in claim 32, wherein all parameters of the ECG device and the ongoing ECG recording may be displayed via the application software program.

45. The assembly as claimed in claim 32, wherein all parameters of the ECG device can be changed and/or corrected during the ongoing ECG recording using the application software program.

46. The assembly as claimed in claim 32, wherein the application software program is designed to be able to interrupt the ECG recording at any time and subsequently carry out a quality check of the ECG.

47. The assembly as claimed in claim 32, wherein error messages generated by the ECG device are displayed and indicated via the application software program.

48. The assembly as claimed in claim 32, wherein the ECG device includes control element which enables switching the ECG device on and off, and that enables a patient to set a marking in the ECG at a desired point in time during the recording of an ECG.

49. The assembly as claimed in claim 32, wherein the application software program is designed to carry out a checking function with respect to a correct contacting of the electrodes of an ECG cable with the human body of a patient and/or to indicate the result of such a checking function.

50. The assembly as claimed in claim 32, wherein the application software program comprises a device management which allows the detection of one or multiple ECG devices.

51. The assembly as claimed in claim 32, wherein the application software program is designed to select the type of the battery to be used during a recording, as a parameter of the ECG device (1).
